(19) United States
(12) Patent Application Publication
(10) Pub. No.: US 2018/0360431 A1
(43) Pub. Date: Dec. 20, 2018

(54) FEEDBACK APPARATUS

(71) Applicant: Micromass UK Limited, Wilmslow (GB)

(72) Inventors: Richard Threlfall, Gatley, Cheadle (GB); Keith George Richardson, New Mills, High Peak, Derbyshire (GB); Steven Derek Pringle, Hoddlesden, Darwen (GB); Michael Raymond Morris, Hadfield, Glossop (GB)

(73) Assignee: Micromass UK Limited, Wlimslow (GB)

(21) Appl. No.: 16/061,275

(22) PCT Filed: Dec. 9, 2016
(86) PCT No.: PCT/GB2016/053880

§ 371 Date: Jun. 11, 2018

(30) Foreign Application Priority Data
Dec. 11, 2015 (GB) 1521848.0

(51) Int. Cl. A61B 10/02; A61B 17/32; A61B 18/14

(52) U.S. Cl. CPC A61B 10/02; A61B 2017/00022; A61B 18/1402; A61B 17/320068

(57) ABSTRACT

A feedback apparatus (22) is provided for use with a system (10) that comprises an analyser (16) and a sampling device (12). The feedback apparatus (22) comprises a receiver (20) that receives from the analyser 16 information relating to a sample obtained using the sampling device (12). The feedback apparatus (22) comprises a feedback device that provides an indication based on the information to an operator (14) of the sampling device (2) whilst the sampling device (12) is within the operator's visual field.

### CROSS-REFERENCE TO RELATED APPLICATION

This application claims priority from and the benefit of United Kingdom patent application No. 1521848.0 filed on 11 Dec. 2015. The entire contents of this application are incorporated herein by reference.

### FIELD OF THE INVENTION

The present invention relates to feedback apparatus and methods of providing feedback. Various embodiments relate to apparatus and methods that make use of information obtained via mass spectrometry.

### BACKGROUND

Rapid evaporative ionization mass spectrometry (“REIMS”) is a technology which has recently been developed for the real-time identification of substrates, for example for the identification of biological tissues during surgical interventions. REIMS analysis of biological tissues has been shown to yield phospholipid profiles having high histological and histopathological specificity, similar to Matrix Assisted Laser Desorption Ionisation (“MALDI”), Secondary Ion Mass Spectrometry (“SIMS”) and Desorption Electrospray Ionisation (“DESI”) imaging.

Coupling of REIMS technology with handheld sampling devices has resulted in iKnife sampling technology, which can provide intra-operative tissue identification. This technology allows surgeons to resect target tissues more efficiently, such as tumours, intra-operatively by providing information that can assist a surgeon in minimizing the amount of healthy tissue removed whilst helping to resect the target tissue. iKnife sampling technology can also be used by non-surgical operators in non-surgical procedures to isolate target matter from an in vitro substrate.

In a known iKnife sampling system, a mass spectrometric signal is obtained by subjecting a substrate to alternating electric current at radiofrequency which causes localized Joule-heating and the disruption of cells along with desorption of charged and neutral particles. The resulting aerosol (e.g., “surgical smoke”) is then transported to a mass spectrometer for on-line mass spectrometric analysis.

Mass spectrometers are usually bulky devices that are located away from the substrate being sampled. Information obtained using a REIMS system with iKnife sampling is typically provided to the operator via a standard display screen connected to the mass spectrometer. This can mean that the operator has to divert his or her attention away from the sample in order to review the information that is being obtained using that system, which can slow down the procedure and lead to errors being made. For example, when it is desired to resect unhealthy target tissue in a human subject, healthy tissue may be removed or some of the unhealthy target tissue may be left behind.

It is desired to provide an improved feedback apparatus and method of providing feedback.

### SUMMARY

According to an aspect there is provided a feedback apparatus for use with a system that comprises an analyser and a sampling device, the feedback apparatus comprising:

a receiver arranged and adapted to receive from the analyser information relating to a sample of a substrate obtained using the sampling device; and

a feedback device arranged and adapted to provide an indication based on the information to an operator of the sampling device whilst the sampling device is within the operator's visual field.

As discussed above, a problem with the known arrangements is that the operator of a sampling device, such as an electrosurgical device, typically has to divert his or her attention away from that device in order to review information that is being obtained using the device. This can slow down the procedure and lead to errors being made. Embodiments address this problem by proving a feedback apparatus that provides an indication to the operator which is based on the information relating to the sample received from an analyser, wherein the indication is provided to the operator whilst the sampling device is within the operator's visual field. The operator therefore does not need to divert his or her attention away from the sampling device in order to react to the information. This can speed up the procedure and reduce the risk of errors being made. Embodiments therefore have significant advantages over the known arrangements.

The feedback apparatus may be (e.g., physically) remote, separate or distal from the analyser. The receiver may comprise a wireless receiver that is arranged and adapted to receive the information wirelessly from the analyser. The receiver may comprise a short-range wireless receiver. The receiver may be selected from the group consisting of: (i) a Wi-Fi receiver; (ii) a Bluetooth receiver; (iii) a Bluetooth Low Energy receiver; and (iv) an Infra-Red receiver. The information that is received may be in the form of one or more signals, such as one or more electrical signals, optical signals, electro-magnetic signals, or wireless signals.

The feedback apparatus may be arranged and adapted to be worn and/or carried by the operator of the sampling device. In some embodiments, the feedback apparatus forms (e.g., an integral) part of or is mountable or mounted to the sampling device. In other embodiments, the feedback apparatus does not form (e.g., an integral) part of the sampling device and/or is (e.g., physically) remote or separate from the sampling device.

The feedback device may be arranged and adapted to provide the indication to the operator whilst the sampling device is both within the operator's visual field and at or adjacent to the site from which the sample of the substrate was or is being obtained. In some embodiments, the feedback device may be arranged and adapted to provide the indication to the operator whilst the sampling device is both within the operator's visual field and being used to obtain further samples of the substrate. However, in other embodiments, the feedback device may be arranged and adapted to provide the indication to the operator whilst the sampling device is within the operator's visual field but is not being used to obtain further samples of the substrate, although the sampling device may still be both within the operator's visual field and at or adjacent to the site from which the sample was obtained. In some embodiments, the feedback device may be arranged and adapted to provide the indication to the operator whilst the sampling device is within the operator's visual field but before the sampling device is used to obtain further samples of the substrate.

The indication may comprise an indication of the type, state, identity and/or composition of the substrate that was or is being sampled. The indication may comprise an indication as to whether the sample relates to target or non-target matter.

The indication may comprise a binary, quantised, graduated, quantitative, qualitative and/or classifying indication based on the information. For example, a first or more intense indication may be provided when the sample relates to target matter and/or a second (different), less intense, or no indication may be provided when the sample relates to non-target matter, or vice versa.

The target matter may comprise or consist of unhealthy and/or unwanted matter, such as contaminated, cancerous, tumorous, malignant, diseased, septic, infected and/or abnormal matter, and the non-target matter may comprise or consist of healthy and/or wanted matter, such as uncontaminated, non-cancerous, non-tumorous, benign, disease-free, aseptic, uninfected and/or normal matter, or vice versa.

The substrate may comprise or consist of organic and/or biological matter. The substrate may comprise or consist of cellular matter such as cellular biomass. The substrate may comprise or consist of biological tissue. The substrate may comprise or consist of food matter. The substrate may comprise or consist of matter belonging to, or that belonged to, a human, animal, plant or fungus (including yeast and/or mould). The substrate may comprise or consist of matter that is living, deceased, or non-living. The substrate may be in vivo or in vitro.

The feedback device may comprise a haptic feedback device. The haptic feedback device may be arranged and adapted to provide a haptic indication to the operator based on the information. The haptic feedback device may be arranged and adapted to be worn and/or carried by the operator of the sampling device. The haptic feedback device may be (e.g., physically) remote or separate from the sampling device, for example so as to avoid disturbing the sampling device. The haptic indication may comprise one or more indications provided by way of: (i) vibration; (ii) pressure; (iii) heat; and (iv) electrical stimulation. The haptic indication may comprise one or more particular temporal or spatial haptic patterns. The haptic feedback device may comprise a device selected from the group consisting of: (i) a vibration device; (ii) a thermal (heating/cooling) device; (iii) a pressure (cuff or band) device; and (iv) an electrical stimulation device.

The feedback device may also or instead comprise a visual feedback device. The visual feedback device may be arranged and adapted to provide a visual indication to the operator based on the information. The visual indication may be within the operator's visual field whilst the sampling device is within the operator's visual field. The visual indication may be, or may appear to the operator to be, provided on or adjacent to the sampling device, for example at or adjacent to a working end of the sampling device, and/or provided on or adjacent to the substrate. The visual indication may comprise one or more indications selected from the group consisting of: (i) an alpha-numeric indication, such as one or more particular values, words or phrases; (ii) a graphical indication, such as one or more particular spatial or temporal patterns or symbols (e.g., regular or irregular shapes); and (iii) a chromatic indication, such as one or more particular colours.

The visual feedback device may comprise an optical device. The visual indication may be superimposed or superposed onto an image that contains the sampling device as seen by the operator through the optical device. The visual indication may be within the field of view of the optical device. The optical device may be arranged and adapted to be worn and/or carried by the operator of the sampling device. The optical device may be (e.g., physically) remote or separate from the sampling device. The visual indication may be provided on a lens of the optical device or projected into the eye(s) of the operator. The optical device may be selected from the group consisting of: (i) spectacles or glasses; (ii) smart glasses; (iii) safety glasses or goggles (iv) a lens or eyepiece; (v) loupes, (vi) a magnifying device; (vii) a microscope; and (viii) an endoscope.

The visual feedback device may also or instead comprise a projector. The visual indication may be, or may appear to the operator to be, projected onto or adjacent to the sampling device, for example onto or adjacent to a working end of the sampling device, and/or projected onto or adjacent to the substrate. The projector may be mountable on or mounted on the sampling device and/or may form (e.g., an integral) part of the sampling device. The projector may be a laser projector.

The visual feedback device may also or instead comprise a visual display device. The visual indication may be displayed by the visual display device. The visual display device may comprise one or more lights (e.g., LEDs) and/or one or more visual display screens. The visual display device may be mountable on or mounted on the sampling device and/or may form (e.g., an integral) part of the sampling device.

In some embodiments, the feedback device may also or instead comprise an audible feedback device. The audible feedback device may be arranged and adapted to provide an audible indication to the operator based on the information. The audible indication may comprise one or more particular sounds, such as alarms, clicks, bleeps, spoken values, spoken words, and/or spoken phrases. The audible feedback device may be arranged and adapted to be worn and/or carried by the operator of the sampling device. The audible feedback device may comprise a personal audible feedback device for the operator, for example comprising one or more speakers or earphones for the operator. In some embodiments, the audible feedback device may be mountable on or mounted on the sampling device and/or may form (e.g., an integral) part of the sampling device. In other embodiments, the audible feedback device may be (e.g., physically) remote or separate from the sampling device.

However, in other embodiments, the feedback device may not comprise an audible feedback device and/or may not be arranged and adapted to provide an audible indication to the operator based on the information. This can be useful when such an audible indication may be overwhelmed by, or may overwhelm, other sounds produced during a procedure, such as important instructions or discussions between personnel and/or important sounds from other devices. Indeed, in some embodiments, the system as a whole does not comprise an audible feedback device and/or is not arranged and adapted to provide an audible indication to the operator based on the information for these reasons.

The feedback apparatus may be provided as part of or separately from a system that comprises the analyser and/or sampling device as described herein in any aspect or embodiment.

The analyser may be arranged and adapted to receive the sample of substrate from the sampling device. The analyser may comprise a (e.g., vacuum) chamber arranged and adapted to receive the sample of substrate from the sampling device.

The analyser may be (e.g., physically) connected or connectable to the sampling device, for example via tubing. The tubing may form part of the analyser and/or sampling device. Receiving the sample may comprise transporting or conveying the sample from the sampling device to the analyser, for example through the tubing. The analyser and/or sampling device may be arranged and adapted to transport or convey the sample from the sampling device to the analyser. Transporting or conveying the sample from the sampling device to the analyser may comprise applying a pressure difference between the analyser and the sampling device, for example between the ends of the tubing. The analyser and/or sampling device may be arranged and adapted to apply the pressure difference. The pressure difference may be applied using a (e.g., venturi) pump and/or vacuum.

The sample may be in the form of an aerosol, smoke, vapour or droplets such as surgical smoke or a droplet stream.

The analyser may be arranged and adapted to analyse the sample so as to generate the information relating to the sample. The analyser may comprise a control system arranged and adapted to control the analyser so as to analyse the sample. The control system may comprise processing circuitry configured to control the analyser so as to analyse the sample.

The analyser may comprise a spectrometer and/or mass analyser, for example a mass spectrometer. The system may comprise or form part of a REIMS system. Analysing the sample may comprise the analyser ionising at least some of the sample to form analyte ions. As discussed above, the sample may be in the form of an aerosol, smoke, vapour or droplets. Thus, the analyser may be arranged and adapted to ionise at least some of the aerosol, smoke, vapour or droplets to form analyte ions. Analysing the sample may comprise forming product (e.g., fragment and/or reaction) ions from the analyte ions. Analysing the sample may comprise separating the analyte ions and/or product ions according to mass, mass to charge, charge state and/or ion mobility. Thus, analysing the sample may comprise (e.g., mass) analysing the analyte ions and/or product ions. Analysing the sample may comprise producing a (e.g., mass) spectrum from the sample, for example from the analyte ions and/or product ions.

Analysing the sample may comprise determining the type, sub-type, state, identity and/or composition of the substrate that was or is being sampled. The type, state, identity and/or composition of the substrate that was or is being sampled may be determined (e.g., automatically) from a (e.g., mass) spectrum. Analysing the sample may comprise determining whether the sample relates to target matter or non-target matter. Whether the sample relates to target matter or non-target matter may be determined (e.g., automatically) from a (e.g., mass) spectrum.

The analyser may be arranged and adapted to receive the sample from the sampling device and/or analyse the sample whilst the sampling device is at or is adjacent to the site from which the sample of substrate was or is being obtained. In some embodiments, the analyser may be arranged and adapted to receive the sample from the sampling device and/or analyse the sample whilst the sampling device is being used to obtain further samples of the substrate. However, in other embodiments, the analyser may be arranged and adapted to receive the sample from the sampling device and/or analyse the sample whilst the sampling device is not being used to obtain further samples of the substrate, although the sampling device may still be at or adjacent to the site from which the sample of substrate was obtained. In some embodiments, the analyser may be arranged and adapted to receive the sample from the sampling device and/or analyse the sample before the sampling device is used to obtain further samples of the substrate.

The analyser may be arranged and adapted to transmit the information to the receiver of the feedback apparatus. The analyser may comprise a transmitter that is arranged and adapted to transmit the information to the receiver of the feedback apparatus. As discussed above, the analyser may be (e.g., physically) remote or separate from the feedback apparatus. The transmitter may comprise a wireless transmitter that is arranged and adapted to transmit the information wirelessly to the receiver of the feedback apparatus. The transmitter may comprise a short-range wireless transmitter. The transmitter may be selected from the group consisting of: (i) a Wi-Fi transmitter; (ii) a Bluetooth transmitter; (iii) a Bluetooth Low Energy transmitter; and (iv) an Infra-Red transmitter. The information that is transmitted may be in the form of one or more signals, such as one or more electrical signals, optical signals, electro-magnetic signals, or wireless signals.

The analyser may be arranged and adapted to transmit the information relating to the sample to the receiver of the feedback apparatus whilst the sampling device is at or is adjacent to the site from which the sample of substrate was or is being obtained. In some embodiments, the analyser may be arranged and adapted to transmit the information to the receiver of the feedback apparatus whilst the sampling device is being used to obtain further samples of the substrate. However, in other embodiments, the analyser may be arranged and adapted to transmit the information to the receiver of the feedback apparatus whilst the sampling device is not being used to obtain further samples of the substrate, although the sampling device may still be at or adjacent to the site from which the sample of substrate was obtained. In some embodiments, the analyser may be arranged and adapted to transmit the information to the receiver of the feedback apparatus before the sampling device is used to obtain further samples of the substrate.

The sampling device may be arranged and adapted to obtain and/or generate the sample of substrate. The sampling device may be an instrument or tool. The sampling device may be hand-held or remote-controlled. The sampling device may be a surgical device. The sampling device may be an electrosurgical device such as a Bovie, a diathermy device such as a diathermy knife, an ultrasonic device such as a cavitation ultrasound surgical aspirator (CUSA), or a laser device such as a laser knife or scalpel.

The sampling device may comprise a working end. The working end may be arranged and adapted to obtain the sample of substrate. The working end may comprise a blade, a scalpel, a lancet, a probe, a protrusion, forceps, an electrode, a transducer, and/or a laser emitter. Obtaining and/or generating the sample may comprise one or more of cutting, resecting, isolating, removing, coagulating, desiccating, fulgurating and cauterising all or part of the substrate. The working end may be arranged and adapted to cut, resect, isolate, remove, coagulate, desiccate, fulgurate and/or cauterise all or part of the substrate.

As discussed above, the sample may be in the form of an aerosol, smoke, vapour or droplets. Obtaining and/or generating the sample may comprise generating an aerosol, smoke, vapour or droplets from the substrate. The sampling device (e.g., the working end of the sampling device) may be arranged and adapted to generate an aerosol, smoke, vapour or droplets from the substrate. The sampling device may be arranged and adapted to pass the aerosol, smoke, vapour or droplets to tubing for transportation or conveyance of the aerosol, smoke, vapour or droplets to the analyser. The aerosol, smoke, vapour or droplets may be ionised as or after the aerosol, smoke, vapour or droplets is/are being generated. The aerosol, smoke, vapour or droplets may be ionised by (e.g., the working end of) the sampling device and/or by causing the aerosol, smoke, vapour or droplets to impact upon a collision surface provided within a vacuum chamber of a mass spectrometer.

In some embodiments, generating the aerosol, smoke, vapour or droplets may comprise applying an alternating or RF electric current to the substrate. The alternating or RF electric current may be pulsed. The sampling device (e.g., the working end of the sampling device) may be arranged and adapted to apply the alternating electric current to the substrate. The alternating electric current may be applied at radiofrequency. In other embodiments, generating the aerosol, smoke, vapour or droplets may comprise applying ultrasound to the substrate. The sampling device (e.g., the working end of the sampling device) may be arranged and adapted to apply the ultrasound to the substrate. In other embodiments, generating the aerosol, smoke, vapour or droplets may comprise directing a laser emitter at the substrate. The sampling device (e.g., the working end of the sampling device) may be arranged and adapted to be directed at the substrate.

According to another aspect there is provided a method of providing feedback comprising:

receiving from an analyser information relating to a sample of a substrate obtained using a sampling device; and

providing an indication based on the information to an operator of the sampling device whilst the sampling device is within the operator's visual field.

The method may further comprise receiving the sample from the sampling device at the analyser. The method may further comprise analysing the sample so as to generate the information relating to the sample. The method may further comprise obtaining and/or generating the sample using the sampling device.

In some embodiments, the method does not encompass treatment of a human or animal body by surgery or therapy and/or does not include diagnosis practiced on a human or animal body. Thus, the method may be non-surgical and/or non-therapeutic. However, in other embodiments, the method may encompass treatment of a human or animal body by surgery or therapy and/or does encompass diagnosis practiced on a human or animal body. Thus, the method may be surgical and/or therapeutic.

According to another aspect there is provided a method of pathology, surgery, treatment, diagnosis, biopsy and/or autopsy comprising a method of providing feedback as described herein in any aspect or embodiment.

Even if not explicitly stated, the methods of providing feedback described herein may comprise performing any step or steps performed by the feedback apparatus, analyser, sampling device, and/or system as described herein in any aspect or embodiment, as appropriate. Similarly, even if not explicitly stated, the feedback apparatus, analyser, sampling device, and/or system described herein may be arranged and adapted to perform any functional step or steps as described herein in any aspect or embodiment, as appropriate.

### BRIEF DESCRIPTION OF THE DRAWINGS

Various embodiments will now be described, by way of example only, and with reference to the accompanying drawings in which:

FIG. 1 shows a system comprising a feedback apparatus according to an embodiment, wherein the feedback apparatus comprises a haptic feedback device that is carried by an operator of the sampling device;

FIG. 2A shows a method of providing feedback to an operator of a sampling device using a feedback apparatus according to an embodiment, wherein the feedback apparatus comprises a projector that forms part of the sampling device;

FIG. 2B shows a method of providing feedback to an operator of a sampling device using a feedback apparatus according to an alternative embodiment, wherein the feedback apparatus comprises a projector that forms part of the sampling device;

FIG. 3A shows a method of providing feedback to an operator of a sampling device using a feedback apparatus according to an embodiment, wherein the feedback apparatus comprises a projector that is separate from the sampling device;

FIG. 3B shows a method of providing feedback to an operator of a sampling device using a feedback apparatus according to an alternative embodiment, wherein the feedback apparatus comprises a projector that is separate from the sampling device;

FIG. 4 shows a system comprising a feedback apparatus according to an embodiment, wherein the feedback apparatus comprises an optical device that is worn by an operator of the sampling device;

FIG. 5 shows a system comprising a feedback apparatus according to an embodiment, wherein the feedback apparatus comprises a visual display device provided on the sampling device; and

FIG. 6 shows a system comprising a feedback apparatus according to an embodiment, wherein the feedback apparatus comprises an audible feedback device that is carried by an operator of the sampling device.

### DETAILED DESCRIPTION

As will be described in more detail below, the various embodiments relate to a feedback apparatus for use with a system that comprises an analyser and a sampling device. The feedback apparatus comprises a receiver arranged and adapted to receive from the analyser information relating to a sample of a substrate obtained using the sampling device. The feedback apparatus also comprises a feedback device arranged and adapted to provide an indication based on the information to an operator of the sampling device whilst the sampling device is within the operator's visual field.

A problem with the known arrangements is that the operator of a sampling device, such as an electrosurgical device, typically has to divert his or her attention away from that device in order to review information that is being obtained using the device. This can slow down the procedure and lead to errors being made.

Embodiments disclosed herein address this problem by proving a feedback apparatus that provides an indication to the operator which is based on the information relating to the sample received from an analyser, wherein the indication is provided to the operator whilst the sampling device is within the operator's visual field. The operator therefore does not need to divert his or her attention away from the sampling device in order to react to the information. This can speed up the procedure and reduce the risk of errors being made. Embodiments therefore have significant advantages over the known arrangements.

The feedback apparatus may be (e.g., physically) remote, separate or distal from the analyser. The receiver may comprise a wireless receiver that is arranged and adapted to receive the information wirelessly from the analyser. The receiver may comprise a short-range wireless receiver. The receiver may be selected from the group consisting of: (i) a Wi-Fi receiver; (ii) a Bluetooth receiver; (iii) a Bluetooth Low Energy receiver; and (iv) an Infra-Red receiver. The information that is received may be in the form of one or more signals, such as one or more electrical signals, optical signals, electro-magnetic signals, or wireless signals.

Various embodiments will now be described in more detail.

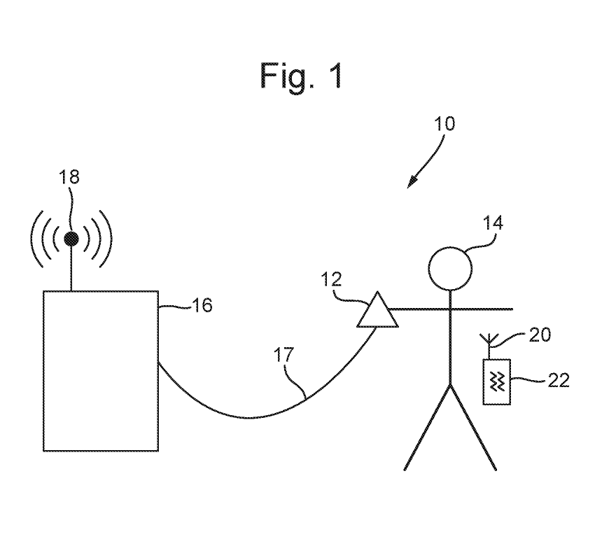

FIG. 1 shows a system 10 comprising a sampling device 12. In this embodiment, the sampling device 12 can include an electrosurgical instrument which can, in some embodiments, include an iKnife sampling device. The sampling device 12 has a working end in the form of a tip, probe or blade that can be used by an operator 14 to resect tissue or a substrate. The tissue or substrate may, for example, comprise cellular biomass belonging to a live human subject undergoing surgery. However, other embodiments are contemplated wherein the substrate may be either in vivo or in vitro and/or may be non-human and non-animal. At the same time as resecting the substrate, the working end of the sampling device 12 generates an aerosol sample of the substrate in the form of surgical smoke, e.g., by applying an alternating electric current to the substrate at radiofrequency. For example, the AC or RF voltage applied to the substrate may be pulsed.

The sampling device 12 may be connected to an analyser 16 via tubing 17. The analyser 16 transports or conveys the aerosol sample from the sampling device 12 to the analyser 16 by applying a vacuum to one end of the tubing 17. The analyser 16 comprises a mass spectrometer that forms part of a REIMS system. The analyser 16 receives the aerosol sample from the sampling device 12, ionises the aerosol sample (e.g., by impact ionisation with a collision surface located within a vacuum chamber of a mass spectrometer) to form analyte ions, and then mass analyses the analyte ions to produce one or more mass spectra. The analyser 16 then generates information relating to the sample by automatically determining from the one or more mass spectra the type of substrate that was sampled and whether the sample relates to target matter or non-target matter. In this embodiment, the target matter may be cancerous or malignant tissue of a human subject.

In some embodiments, the analyser 16 can further comprise a wireless transmitter 18 that transmits the information about the sample to a wireless receiver 20 of a remote feedback apparatus 22.

In some embodiments, such as the embodiment that is shown in FIG. 1, the feedback apparatus 22 can include a haptic feedback device worn by the operator 14 that can provide a vibrating indication to the operator 14 based on the information. In other embodiments, the sampling device 12 can include a haptic feedback device and/or receiver of the feedback apparatus. For example, the haptic feedback device and/or receiver of the feedback apparatus can be built into the handle of the sampling device, attached to the sampling device, or otherwise associated with the sampling device.

In some embodiments, the haptic feedback indication can be binary, e.g., a vibration is not provided when the sample relates to target matter and a vibration is provided when the sample relates to non-target matter. In further embodiments, the haptic feedback indication can provide additional information about the sample. For example, the vibration or other haptic feedback can vary, e.g., in frequency or intensity, to provide an indication of whether the aerosol sample relates to target matter or non-target matter.

In embodiments in which the indication is haptic, the indication can be provided to the operator 14 without the sampling device 12 leaving the operator's visual field. The operator 14 therefore does not need to divert his or her attention away from the sampling device 12 in order to react to the information. For example, the operator 14 can move the sampling device 12 towards or through target matter and/or away from non-target matter based on the indication. This can speed up the procedure and reduce the risk of errors being made.

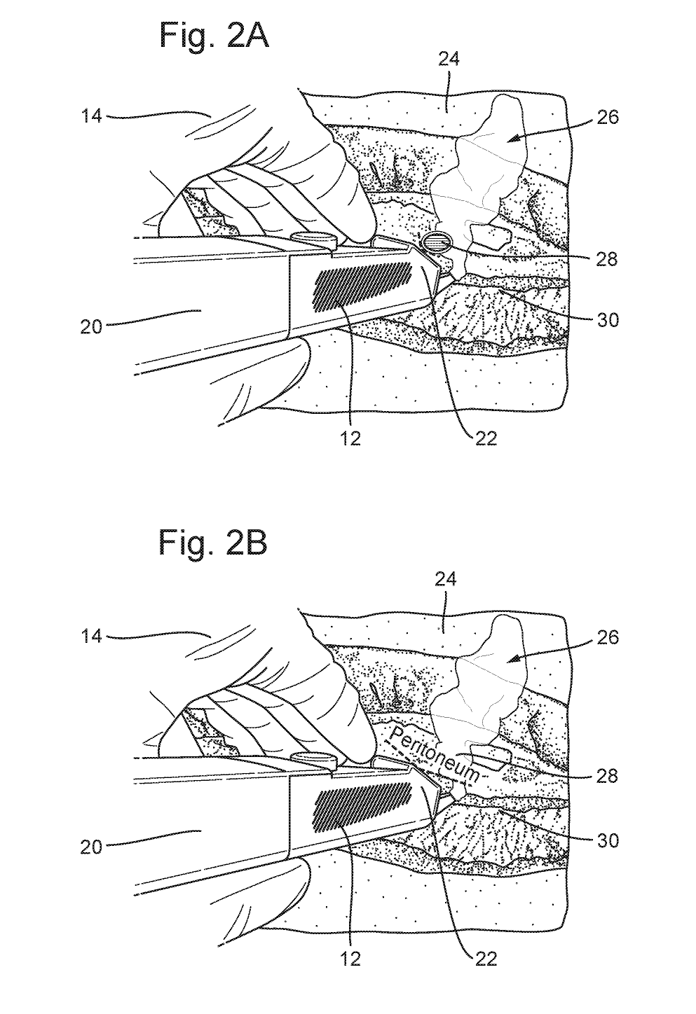

FIG. 2A shows a method of providing an indication to an operator 14 of a sampling device 12 according to an alternative embodiment. In this embodiment, rather than a haptic feedback device, the feedback apparatus 22 can comprise a visual feedback device that forms part of the sampling device 12 (the receiver 20 can also form part of the sampling device 12 as is shown in FIG. 2A, or may be separate from the sampling device). For example, visual feedback can be provided by a visual feedback device in the form of a projector that forms part of the sampling device 12. The feedback device can project a visual indication 28 based on the information received by the receiver 20.

In some embodiments, the visual indication 28 can be in the form of a spot of light, e.g., laser light, LED light, or other light source, that is projected onto the tissue of a human subject 24 that is adjacent to the working end of the sampling device 12. In some embodiments, the indication 28 can be binary in that a light spot is provided when the aerosol sample 26 relates to target matter and a light spot is not provided when the sample relates to non-target matter. In some embodiments, the visual indication can provide additional information about the sample. For example, the spot of light can have a colour that provides an indication of whether the aerosol sample 26 relates to target matter or non-target matter. For another example, the spot of light can change from a first colour to a second colour to provide an indication whether the aerosol sample 26 relates to target matter or non-target matter, e.g., the spot of light can have a first colour when the aerosol sample relates to target matter and a second colour when the aerosol sample relates to non-target matter.

FIG. 2B shows a method of providing an indication to an operator 14 of a sampling device 12 according to a further alternative embodiment. In this embodiment, the feedback apparatus 22 again comprises a visual feedback device in the form of a projector and a receiver 20 that form part of the sampling device 12. The feedback device again projects a visual indication 28 based on the information received by the receiver 20. However, in this embodiment, the visual indication 28 is in the form of a word that is projected onto the tissue of the human subject 24 that is adjacent to the working end of the sampling device 12. In this embodiment, the indication 28 is a classifying indication in that it specifies the type of substrate to which the aerosol sample 26 relates. In this example, the aerosol sample 26 relates to the peritoneum as indicated by the indication 28.

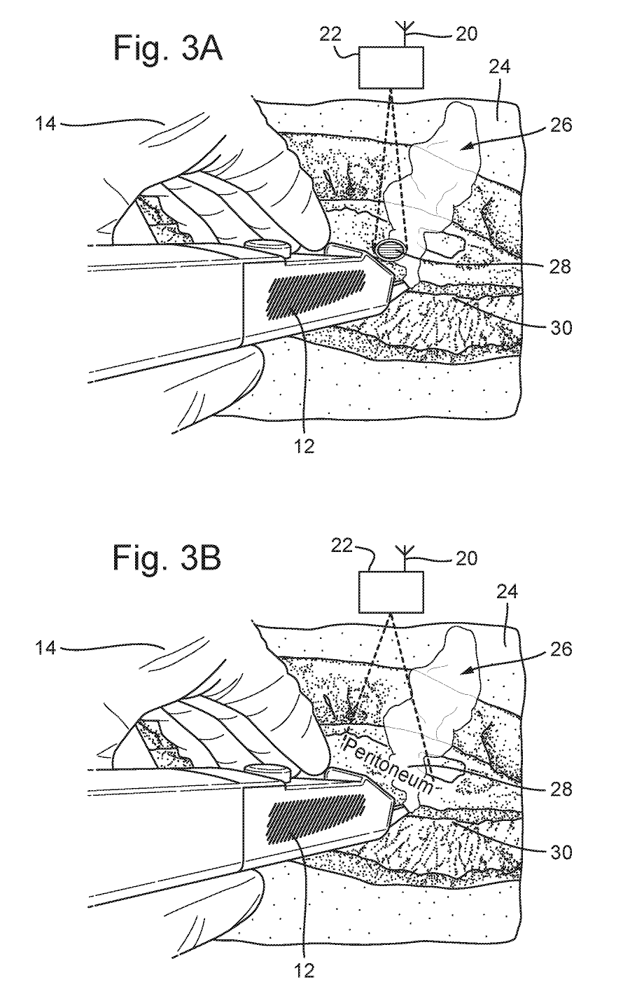

FIGS. 3A and 3B each show a method of providing an indication to an operator 14 of a sampling device 12 according to further embodiments. In these embodiments, the feedback apparatus 22 again comprises a visual feedback device in the form of a projector and a receiver 20. However, rather than the feedback apparatus 22 forming part of the sampling device 12, the feedback apparatus 22 is separate from the sampling device 12. In these embodiments, the projector can be provided above the human subject 24 and arranged or oriented so as to project a visual indication 28 based on the information received by the receiver 20 onto the subject 24.

In some embodiments, such as the embodiment which is shown in FIG. 3A, the visual indication 28 can be in the form of a spot of light, e.g., laser light, LED light, or other light source, that is projected onto the tissue of a human subject 24 that is adjacent to the working end of the sampling device 12. As discussed above, in some embodiments, such an indication 28 can be binary in that a light spot is provided when the aerosol sample 26 relates to target matter and a light spot is not provided when the sample relates to non-target matter. As discussed above, in some embodiments, the visual indication can provide additional information about the sample. For example, the spot of light can have a colour that provides an indication of whether the aerosol sample 26 relates to target matter or non-target matter. For another example, the spot of light can change from a first colour to a second colour to provide an indication whether the aerosol sample 26 relates to target matter or non-target matter, e.g., the spot of light can have a first colour when the aerosol sample relates to target matter and a second colour when the aerosol sample relates to non-target matter.

In other embodiments, such as the embodiment which is shown in FIG. 3B, the visual indication 28 can be in the form of a word that is projected onto the tissue of the human subject 24 that is adjacent to the working end of the sampling device 12. As discussed above, in these embodiments, such an indication 28 can be a classifying indication that specifies the type of substrate to which the aerosol sample 26 relates. In this example, the aerosol sample 26 again relates to the peritoneum as indicated by the indication 28.

In the embodiments shown and described above in relation to FIGS. 2A, 2B, 3A and 3B, since the indication 28 is visible adjacent to the working end of the sampling device 12, the indication is provided to the operator 14 whilst the sampling device 12 is within the operator's visual field. The operator 14 therefore does not need to divert his or her attention away from the sampling device 12 in order to take note of or react to the information. For example, the operator 14 can move the sampling device 12 towards or through target matter and/or away from non-target matter based on the indication 28. This can speed up the procedure and reduce the risk of errors being made.

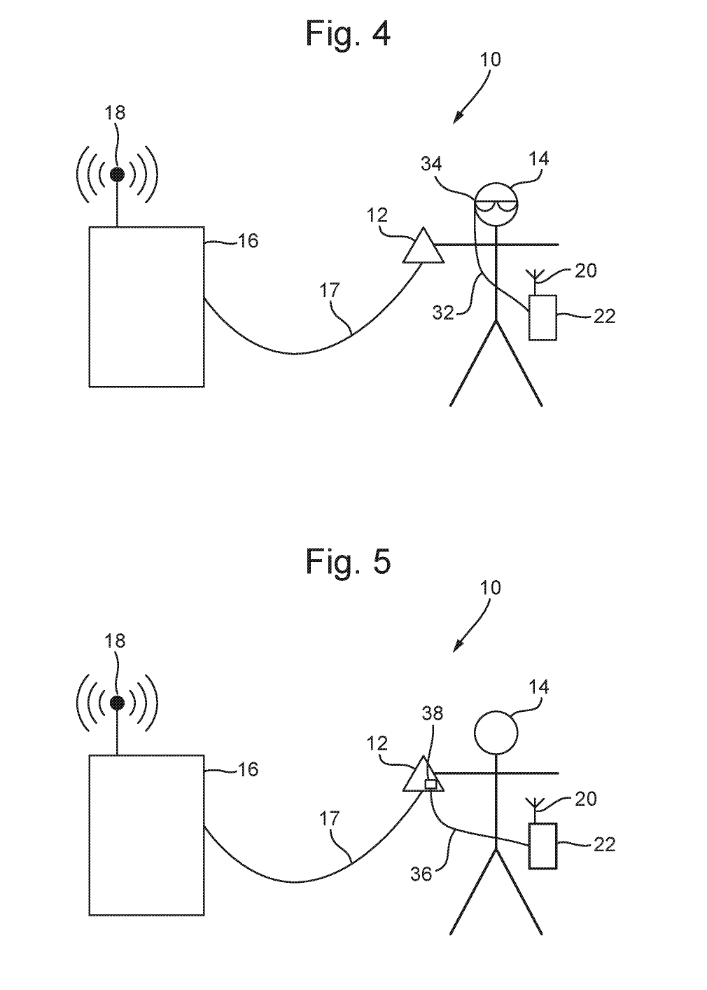

FIG. 4 shows a system 10 comprising a feedback apparatus 22 according to further embodiments. The system 10 in these embodiments can comprise the surgical device 12 and/or analyser 16, etc., as described with reference to FIG. 1. However, in these embodiments, rather than a haptic feedback device, the feedback apparatus 22 can include a visual feedback device that is coupled, e.g., via a cable 32 or wirelessly, to an optical device 34 worn by the operator 14. The visual feedback device can provide a visual indication to the operator 14, via the optical device 34, based on the information relating to the sample. The optical device 34 can be, e.g., spectacles, goggles, loupes, etc., worn by the operator 14. The visual feedback device can provide a visual indication that is superimposed or superposed onto an image that contains the sampling device 12 as seen by the operator 14 through the optical device 34. The visual indication may be superimposed or superposed onto the image by projecting the visual indication onto a lens of the optical device 34 or into the eye of the operator 14. The visual indication may be provided by one or more particular colours, patterns, symbols, values, words and/or phrases.

In these embodiments in which the indication is within the operator's visual field as seen through the optical device 34, the indication can again be provided to the operator 14 without the operator 14 needing to divert his or her attention away from the sampling device 12.

FIG. 5 shows a system 10 comprising a feedback apparatus 22 according to further embodiments. The system 10 in these embodiments can comprise the surgical device 12 and/or analyser 16, etc., as described with reference to FIG. 1. However, in these embodiments, rather than a haptic feedback device, the feedback apparatus 22 can include a visual feedback device that is coupled, e.g., via a cable 36 or wirelessly, to a visual display device 38 provided on the surgical device 12. The visual feedback device can provide a visual indication to the operator 14, via the visual display device 38, based on the information relating to the sample. The visual display device 38 may comprise, e.g., one or more lights, such as LEDS, and/or one or more visual display screens. The visual indication may be provided by one or more particular colours, patterns, symbols, values, words and/or phrases.

In other embodiments, in addition to the visual display device 38, the sampling device 12 can include the receiver. For example, the receiver can be built into the handle of the sampling device 12, attached to the sampling device 12, or otherwise associated with the sampling device 12.

In these embodiments in which the indication is provided via a visual display device 38 on the sampling device 12, the indication can again be provided to the operator 14 without the operator 14 needing to divert his or her attention away from the sampling device 12.

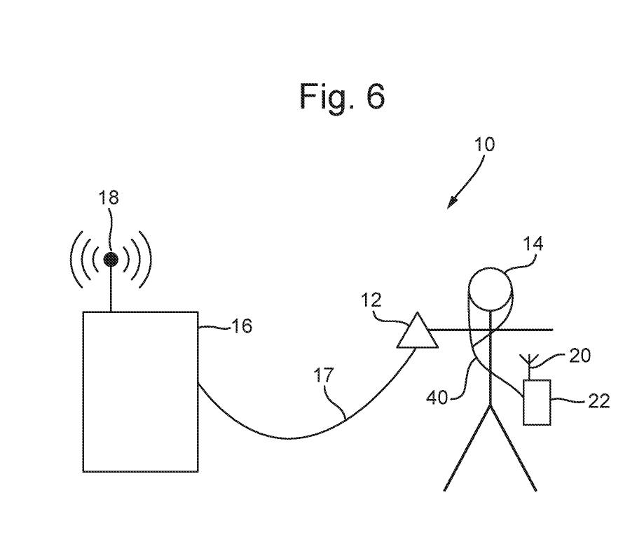

FIG. 6 shows a system 10 comprising a feedback apparatus 22 according to further embodiments. The system 10 in these embodiments can comprise the surgical device 12 and/or analyser 16, etc., as described with reference to FIG. 1. However, in these embodiments, rather than a haptic feedback device, the feedback apparatus 22 can include an audible feedback device, which may be worn or carried by the operator 14. The audible feedback device can provide an audible indication to the operator 14 based on the information relating to the sample. The audible indication may be provided to the operator via headphones or earphones 40 for the operator 14. Alternatively, the audible indication may be provided to the operator via a speaker.

In other embodiments, the sampling device 12 can include an audible feedback device and/or receiver. For example, the audible feedback device and/or receiver can be built into the handle of the sampling device 12, attached to the sampling device 12, or otherwise associated with the sampling device 12. In these embodiments, the audible indication may again be provided to the operator 14 via earphones coupled to the sampling device 12 or via a speaker provided on the sampling device 12.

In some embodiments, the audible feedback indication can be binary, i.e., a sound is not provided when the sample relates to target matter and a sound is provided when the sample relates to non-target matter. In further embodiments, the audible feedback indication can provide additional information about the sample. For example, the sound can vary, e.g., in frequency or intensity, or spoken values, words or phrases may be provided to indicate whether the aerosol sample relates to target matter or non-target matter.

In these embodiments in which the indication is audible, the indication can again be provided to the operator 14 without the operator needing to divert his or her attention away from the sampling device 12. However, in other embodiments, the operator 14 may find that an audible indication is too distracting. Thus, in some embodiments, an audible indication is not provided to the operator 14 but other, non-audible, indications are provided to the operator 14 based on the information, for example, haptic and/or visual indications as discussed above.

It will be apparent that the feedback apparatus according to various embodiments provides significantly improved feedback to a user of a surgical tool and ensures that the feedback is provided to the user within the user's visual field as he or she operates the surgical tool. In particular, the user is able to operate the surgical tool without having their attention diverted away from the surgical site to e.g., a display panel proximal to a remote mass spectrometer.

It will be apparent, therefore, that the various embodiments described above are particularly beneficial.

Although the present invention has been described with reference to various embodiments, it will be understood by those skilled in the art that various changes in form and detail may be made without departing from the scope of the invention as set forth in the accompanying claims.

1. A feedback apparatus for use with a system that comprises an analyser and a sampling device, the feedback apparatus comprising:
a receiver arranged and adapted to receive from the analyser information relating to a sample of a substrate obtained using the sampling device; and
a feedback device arranged and adapted to provide an indication based on the information to an operator of the sampling device whilst the sampling device is within the operator's visual field.

2. A feedback apparatus as claimed in claim 1, wherein the feedback apparatus is remote, separate or distal from the analyser.

3. A feedback apparatus as claimed in claim 1, wherein the receiver comprises a wireless receiver that is arranged and adapted to receive the information wirelessly from the analyser.

4. A feedback apparatus as claimed in claim 1, wherein the feedback apparatus is arranged and adapted to be at least one of worn and carried by the operator of the sampling device.

5. A feedback apparatus as claimed in claim 1, wherein the feedback apparatus forms part of or is mountable to the sampling device.

6. A feedback apparatus as claimed in claim 1, wherein the feedback device is arranged and adapted to provide the indication to the operator whilst the sampling device is both within the operator's visual field and at or adjacent to the site from which the sample of substrate was or is being obtained.

7. A feedback apparatus as claimed in claim 1, wherein the indication comprises an indication of at least one of the type, sub-type, state, identity and composition of the substrate that was or is being sampled.

8. A feedback apparatus as claimed in claim 1, wherein the indication comprises an indication as to whether the sample relates to target matter or non-target matter.

9. A feedback apparatus as claimed in claim 1, wherein the substrate comprises or consists of cellular matter.

10. A feedback apparatus as claimed in claim 1, wherein the substrate comprises or consists of biological tissue.

11. A feedback apparatus as claimed in claim 1, wherein the substrate comprises or consists of food matter.

12. A feedback apparatus as claimed in claim 1, wherein the feedback device comprises a haptic feedback device arranged and adapted to provide a haptic indication to the operator based on the information.

13. A feedback apparatus as claimed in claim 1, wherein the feedback device comprises a visual feedback device arranged and adapted to provide a visual indication to the operator based on the information.

14. A feedback apparatus as claimed in claim 13, wherein the visual feedback device comprises an optical device, the visual indication being superimposed or superposed onto an image that contains the sampling device as seen by the operator through the optical device.

15. A feedback apparatus as claimed in claim 13, wherein the visual feedback device comprises a projector, the visual indication being, or appearing to the operator to be, projected onto or adjacent to at least one of the sampling device and the substrate.

16. A feedback apparatus as claimed in claim 13, wherein the visual feedback device comprises a visual display device, the visual indication being displayed by the visual display device.

17. A feedback apparatus as claimed in claim 1, wherein the feedback device comprises an audible feedback device arranged and adapted to provide an audible indication to the operator based on the information.

18. A feedback apparatus as claimed in claim 1, wherein the feedback device does not comprise an audible feedback device arranged and adapted to provide an audible indication to the operator based on the information.

19. A system comprising:
a sampling device;
an analyser; and
a feedback apparatus, the feedback apparatus comprising:
a receiver arranged and adapted to receive from the analyser information relating to a sample of a substrate obtained using the sampling device; and
a feedback device arranged and adapted to provide an indication based on the information to an operator of the sampling device whilst the sampling device is within the operator's visual field.

20. A method of providing feedback comprising:
receiving from an analyser information relating to a sample of a substrate obtained using a sampling device; and
providing an indication based on the information to an operator of the sampling device whilst the sampling device is within the operator's visual field.
